(19) United States
(12) Patent Application Publication
(10) Pub. No.: US 2021/0212981 A1
(43) Pub. Date: Jul. 15, 2021

(54) CANNABINOID SYSTEMS AND METHODS: WATER-SOLUBILITY, TARGETING, AND AUGMENTATION

(71) Applicants: Michael J. McCarthy, Carlsbad, CA (US); John C. Gustin, Carlsbad, CA (US)

(72) Inventors: Michael J. McCarthy, Carlsbad, CA (US); John C. Gustin, Carlsbad, CA (US)

(73) Assignee: ECS Health Sciences, Inc., Carlsbad, CA (US)

(21) Appl. No.: 16/744,141

(22) Filed: Jan. 15, 2020

(51) Int. Cl. A61K 31/352; A61K 31/05; A61K 47/64

(52) U.S. Cl. CPC A61K 31/352; A23L 2/00; A61K 47/64; A61K 31/05

(57) ABSTRACT

The present invention describes compositions, and associated methods, comprising a cannabinoid operably linked with a biologically active agent for augmented cannabinoid agents.

### BACKGROUND OF THE INVENTION

The present invention is in the technical fields of chemistry, biochemistry and biology. More particularly, the present invention is in the area of functionally activated cannabinoids.

### SUMMARY OF THE INVENTION

Certain embodiments of the present invention provide water-soluble cannabinoids, methods for their manufacture and methods of use.

Certain embodiments provide aqueous Cannabis isolates including solvated cannabinoids (cannabinoids in a water-based solution), methods of manufacture, and methods of use. Certain isolates include water-solvated isolates of hemp, marijuana or combinations thereof.

Certain embodiments provide a cannamide and methods for making and using them.

In certain embodiments, a cannamide comprises a cannabinoid and a polypeptide in combination. In certain preferred embodiments, a cannamide comprises a complex including a cannabinoid operably linked with a polypeptide.

In certain embodiments, a cannamide comprises a cannabinoid operably linked with a polypeptide by an amide bond.

In certain embodiments, a cannamide comprises a cannabinoid operably linked with a polypeptide by a non-covalent bonding.

In certain embodiments, a cannamide comprises a cannabinoid operably linked with a polypeptide by confinement in a complex.

Certain methods embodied in the present invention include the manufacture of a water-based cannabinoid beverage.

Certain methods embodied in the present invention include the preparation and the administration of water-based cannabinoid remedies, medicaments, and therapeutics.

Certain embodiments provide a method of extracting cannabinoids from cannabinoid producing plants, comprising: reacting a water-soluble peptide with a plant biomass under conditions for amide bond formation between complementary amino and carboxylic functional groups on the cannabinoids and the polypeptides and performing aqueous and organic extraction steps to collect cannabinoids separate from the matrix.

### BRIEF DESCRIPTION OF THE DRAWINGS

FIG. 1: The amide bond is synthesized when the carboxyl group of one amino acid molecule reacts with the amino group of the other amino acid molecule, causing the release of a molecule of water (H2O), hence the process is a dehydration synthesis reaction forming an amino acid chain, which is also known as a polypeptide.

FIG. 2: An example of a cannabinoid, in this case cannabidolic acid (CBDA) reacting at a carboxylic acid moiety with a polypeptide containing an amine group (H2N—R2), thus forming a cannamide (comprising a cannabidiol operably linked with a polypeptide polymer in this non-limiting example). The R2 can be any sequence of amino acids desired and thus is very adaptable for including peptides having any range of activity or activities.

FIG. 3: Another example of a cannabinoid in a cannamide formation, in this non-limiting case, the cannabinoid is tetrahydrocannabinolic acid (THCA).

FIG. 4: Exemplary illustration of combinations of components useful in cannamide construction. A cannamide (140) is formed by combination of a cannabinoid (100) in operable linkage (145 for a covalent linkage and 150 with the use of a non-covalent linkage) with one or more of: a solubility factor (110), a targeting factor (115), a bioactivity factor (120), an additional factor of choice (125), for example, an enzyme.

FIG. 5: Exemplary illustration of combinations of components useful in another type of cannamide (200) with construction including the use of a containment substrate or container (210) (such as a networked substance, e.g., a gel or polysaccharide network, or a micro-bead). The containment system includes a cannabinoid (215) and one or more of components selected from: a targeting factor (220) and a solubility factor (222) or, optionally, both a targeting factor (220) and a solubility factor (222). The containment component barrier to movement of the cannamide from inside or within (215) to outside of the containment (235) and the barrier is optionally semipermeable (225) such as with pores (e.g., in a microbead) or spaces (e.g., in a networked substrate) (225). The cannamide within the containment (215) can move through the semipermeable barrier (225 gap/space/pore) to be outside of the containment (235) in embodiments using a time-release or delayed release therapeutic or research reagent.

FIG. 6: Illustration with example of two amino acids and formation of an operable linkage, in this example a covalent, amide bond; thereby forming a dipeptide in a condensation reaction that also releases water as a product.

FIG. 7: Illustration of cannabidiolic acid (CBDA) on the left with an illustration of a cannamide on the right having a CBD compound covalently linked with a peptide through an amide bond, in this non-limited example at the 4′ carbon through condensation reaction and formation of an amide bond between the CBD and the peptide. A polypeptide is optionally operably linked in many positions of a cannabinoid as exemplified by the stars near particularly reactive chemical groups of the cannabinoid. CBDA is the acidic form of cannabidiol (CBD) having a 4′ carboxylic acid moiety in the acid form.

FIG. 8: An example of a cannamide having a synapse targeting peptide operably linked through the 3′ hydroxyl of the cannabidiol (CBD) reactant. A synapse targeting peptide (called R-R-R-R-P-Y-R-G-V-I-S-F) is disclosed in the scientific literature.

FIG. 9: a diagram illustrating selected factors of the endocannabinoid system including receptors, ligands, biosynthetic enzymes, and degrading or elimination enzymes.

FIG. 10: Illustrations of several cannabinoid compounds.

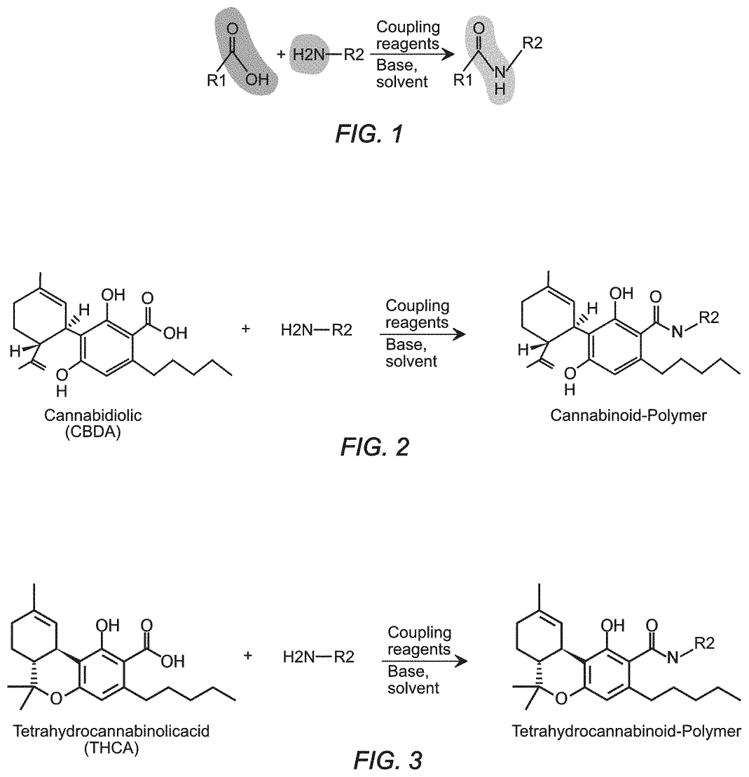

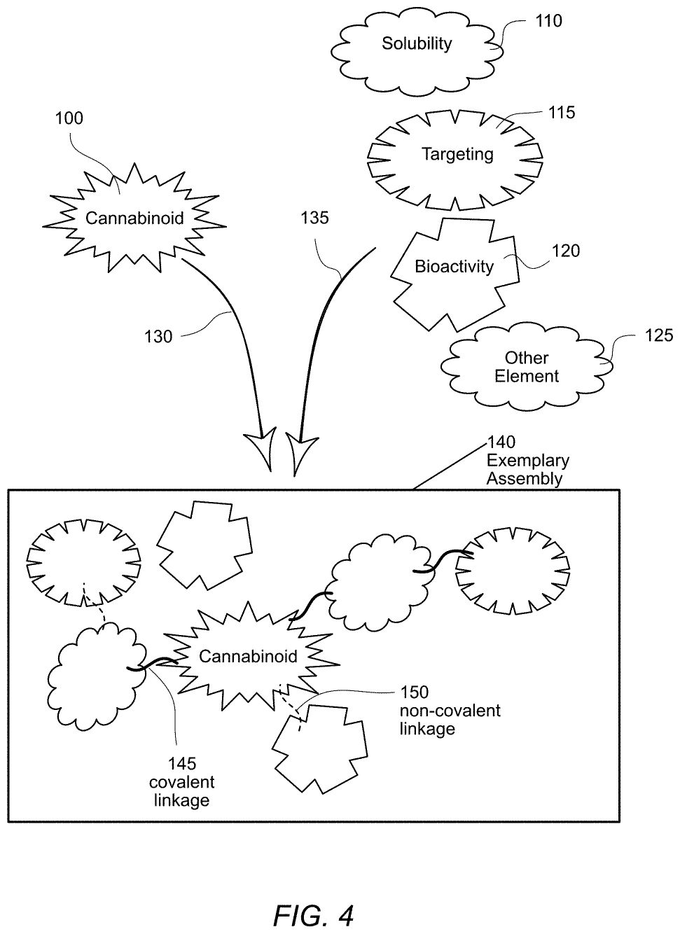

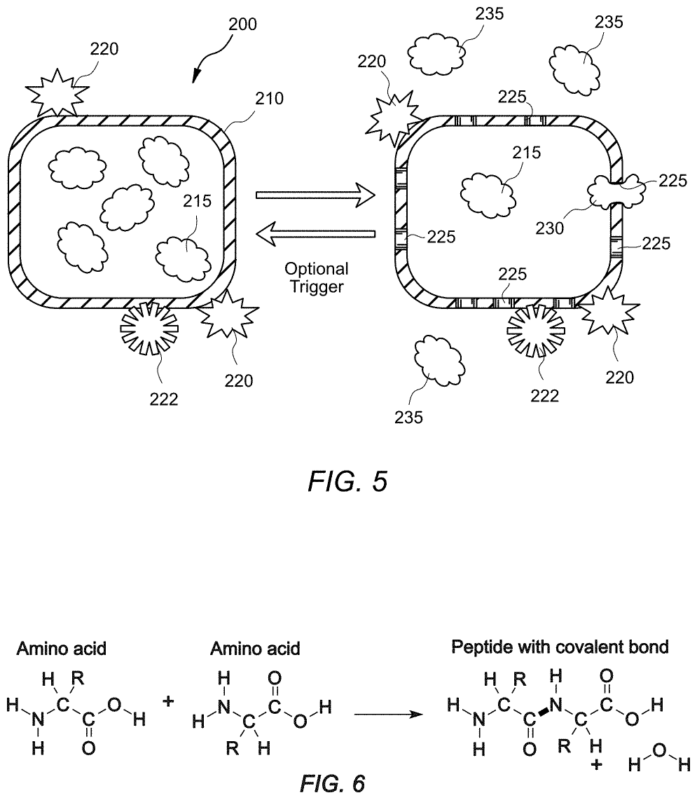

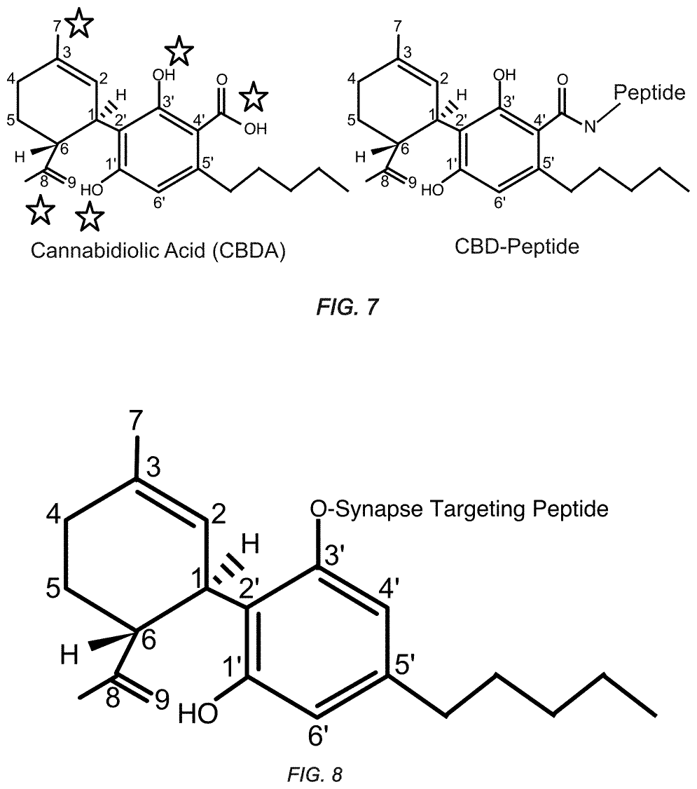

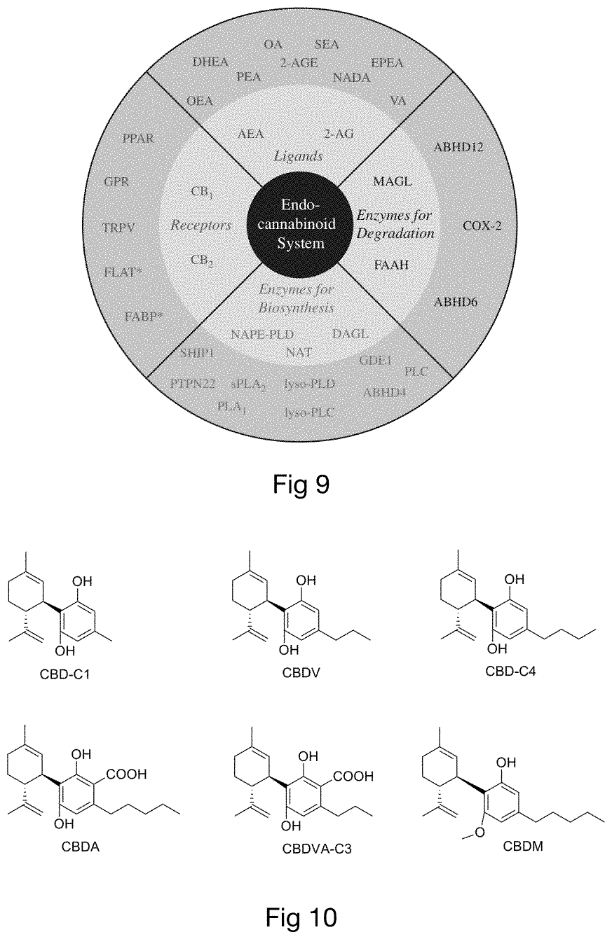

### DETAILED DESCRIPTION OF THE INVENTION

Embodiments of the present invention include compositions, systems, methods of manufacture and methods of use; related to cannabinoid compounds, their features, properties and activities, compositions or systems having enhancements and new-found features; comprising but not limited to: water-solubility, water-compatibility, enhanced or gain of targeting, enhanced or gain of activity, enhanced or gain of bioavailability, or enhanced or gain of disease therapeutic, or any combination thereof.

Certain embodiments provide cannabinoids, and methods for their manufacture, having one or more properties of enhanced solubility in aqueous solution, enhanced cannabinoid receptor binding, enhanced bioavailability, gain of binding activity, gain of bioactivity, gain of biological targeting, gain of biological mobility, or any combination thereof.

### Selected Terms

When “a” is used as the primary antecedent basis to introduce an element, including in the specification and in the claims when introducing a claim element, the “a” is understood to mean “one or more” unless it is specifically modified to indicate otherwise. For example “a cannabinoid” means or refers to one or more cannabinoids. In another example, “a single cannabinoid” refers to exactly one cannabinoid compound or exactly one type of cannabinoid compound. In certain instances the type of cannabinoid is indicating using a more specific name such as tetrahydrocannabinol, cannabidiol, and etc.

An accessory is an item or an element which can be added to something else in order to make that something else more useful, versatile, or attractive. In certain embodiments, a system includes a cannabinoid and an accessory, wherein the system has, or the accessory imparts on the system a structure or use that is enhanced or not found in the cannabinoid(s) separate from the system or accessory.

A composition is a thing composed of two or more elements, constituents, modules or parts.

A system is a set of items working together, for example, as parts of a mechanism or an interconnecting network. The items of the system, as used herein, can be linked in any manner unless the type of linkage is specified.

Nanoscale refers to distances or particle sizes that are less than one micron in any dimension, preferably 1 nm to 500 nm, and more preferably 1 nm to 100 nm.

An aptamer refers to a molecular binding partner of any kind, unless the type of binding partner is indicated. Thus, as used herein, the term aptamer can be more expansive than binding partners that are oligonucleotides, peptides or proteins. For example, as used herein, the term aptamer could include, without limitation, a peptide, a protein, a nucleic acid, an interacting enzyme (having a temporal binding or interaction), a biomarker, a disease biomarker, an antibody, a receptor, a sugar, a carbohydrate, a collagen, a polymer, an inorganic particle, other molecules and biological molecules, or any combination thereof or any molecular combination thereof.

Certain embodiments provide compositions, comprising: a cannabinoid having an enhanced water-solubility, a gain of function in targeting, a gain of a biological activity, or any combination thereof. In certain optional embodiments, the compositions further include one or more terpenoids.

Certain embodiments provide a cannabinoid composition including a cannabinoid and an accessory element, wherein the composition has an enhanced water-solubility, binding affinity, targeting, biological activity, or any combination thereof; when compared to the cannabinoid apart from the accessory element.

Certain embodiments provide a modular system including one or more cannabinoid modules and one or more accessory modules, wherein the system has an enhanced property when compared to the cannabinoid or cannabinoids apart from the system, (i.e., a cannabinoid without an accessory module).

Certain embodiments provide reagents and methods for obtaining one or more cannabinoids from a biomass or a cannabinoid containing extract.

Certain embodiments provide cannabinoids, cannabinoid compositions and cannabinoid systems; methods of manufacture and methods of use thereof.

Systems

Certain embodiments provide a modular assembly, comprising a cannabinoid module and a bioactive module (optionally referred to as an accessory module).

By way of non-limiting and illustrative examples, certain compositions of the present invention provide systems or modular assemblies comprising cannabinoids and accessory factors.

In certain preferred assemblies, a cannabinoid module and a bioactive module are combined by a covalent bond, preferably an amide bond. A method of manufacture of the assembly is the reaction of a carboxyl group (—COOH) of a phytocannabinoid having (or made to have) said carboxyl group with an amide (—NH2) group of an amino acid or N-terminus of a polypeptide via a dehydration synthesis reaction (alternatively known as a condensation reaction).

Certain embodiments provide a system, comprising: a cannabinoid and an accessory, wherein the system has a feature that is not present in the cannabinoid component without the accessory.

Certain embodiments provide a system, comprising: a cannabinoid and an accessory, wherein the system has an enhanced property when compared with the the property of the cannabinoid apart from the system.

Certain preferred embodiments of the present invention provide a modular system, comprising: a cannabinoid module and an accessory module, wherein the system has an enhanced property when compared with the cannabinoid apart from the system.

In certain preferred embodiments, one or more cannabinoid modules are combined with one or more accessory modules, wherein the modules can be mixed and matched to build systems having a desirable feature, property, or combinations thereof. A number of preferred cannabinoids and accessories are described herein and other modules can be combined within the scope of the present invention.

In certain preferred embodiments, one or more cannabinoid modules are combined with one or more accessory modules, wherein the modules can be mixed and matched to build systems having assumed features, structural features, named features, functional features, or a combination of such features. In certain embodiments, the modules comprising an embodied system of the invention have features. Preferably the accessory module(s) provide at least one feature that is not overlapping with a feature of the cannabinoid module(s).

Certain preferred examples of accessory module features include, but are not limited to:
improved water or aqueous solubility (at least 15% or more increase compared to a non-system cannabinoid of the same or comparable type(s), preferably 20% or more, 50% or more, 75% or more, and still more preferably 100% or more),
improved water or aqueous solubility (2 mg or more dissolved per ml of water (or aqueous solution) compared to a non-system cannabinoid of the same or comparable type(s), preferably 3 mg or more per ml of solute, more preferably (in increasing order) 4 mg, 5 mg, 6 mg, 7 mg, 8 mg, 9 mg, 10 mg, 15 mg, 20 mg, 25 mg, 30 mg, 40 mg, 50 mg 100 mg or more per ml of solute (but being more soluble than the isolated cannabinoid)),
a water-compatibility of a system comprising an emulsion, a nanoparticle, or compound delivery particle; such that an increase in the amount, relative amount or molar amount of the cannabinoid(s) of the system is/are present in a water or aqueous solution in comparison to the amount of the isolated cannabinoid(s) in the solution (having similar improvements as listed for water-solubility above), and
accessory module(s) having a named or determined feature including, but not limited to a feature of a targeting, a binding, a bioactivity, a membrane penetrating feature (cell wall, cell membrane, nuclear membrane, cellular organelle membrane, and the like), a endocytosis inducing feature, a receptor binding, a non-cannabinoid receptor binding, a binding to a cannabinoid receptor heterologous to the cannabinoid modules(s) present in the system, an inhibiting, an activating, an enzymatic, and any combination thereof.

In certain embodiments of the modular systems herein, the features do not necessarily require a measurement, such as a measurement of an activity, to define the nature of the system or a component thereof. This is because such systems can be assembled based on named module features (or optionally assumed, hypothesized, expected, or described module features, activities, or the like) and the assembled system is useful as a research tool (for example, to study gain of function cannabinoid compounds and compositions).

For example, such a system is useful for observing a delivery of a cannabinoid to a cell type or receptor that the cannabinoid alone, is not known to associate with. In another example, by selecting an accessory module with a desired property and modifying or combining the cannabinoid with accessory is useful to observe an enhanced function or gain of function of the cannabinoid component. In another example, by selecting an accessory with a desired property and modifying or combining the cannabinoid with an accessory module is useful to observe an enhanced function or gain of function of the cannabinoid component. For example, a cannabinoid module, such as THC, CBG, CBD, or acid form thereof, is combined with a cell penetrating peptide and an enzyme inhibitor of fatty acid amide hydrolase is useful for measuring the movement or kinetics of cellular entry of the modular system and other properties of the system, such as inhibition of FAAH enzyme (in the cell and/or in the bodily fluids such as blood, serum, plasma and the like) and to measure expected properties of the cannabinoid module (as measured apart from the system).

Properties

In certain preferred embodiments, one or more cannabinoid modules are combined with one or more accessory modules, wherein the modules can be mixed and matched to build systems having an enhanced property, a gain of a property, or both. In certain embodiments, the properties can be a combination of the properties of the individual modules. In certain embodiments, the properties can arise from the combination of the modules, wherein the modules of the system do not have the properties when they are isolated, but the property or properties are a result of the combined modules as part of a functional system.

In certain embodiments, a property of a modular system herein, results in a measurable enhancement of an activity, characteristic, availability, concentrating, or other property of the system that is not significantly observed for the cannabinoid module apart from the system. In certain embodiments, the system property is not observed in the cannabinoid apart from the system.

Certain embodiments provide a novel cannabinoid or cannabinoid containing system useful for enhancing or creating an aqueous soluble cannabinoid formulation. Such formulation is useful, in certain exemplary embodiments, for inclusion in (without being limited to): a carbonated beverage, a lozenge, a capsule, a gelatin capsule, a tablet, an oral consumable, or a combination thereof.

In certain embodiments, the cannabinoid or cannabinoid containing system is useful (without limitation) as an ingredient, active ingredient, or adjuvant in a medicament and/or a deliverable for oral nasal, rectal, vaginal, transdermal, injectable, or inhalation administration (or a combination of two or more delivery routes). In certain embodiments, the cannabinoid/system is combined with other ingredients (active, inactive, and/or adjuvants) in a formulation including medicaments.

In certain preferred embodiments, administration to a mammal or person is to that animal or person who is in need of treatment for a disease or symptom of a disease or condition.

In certain preferred embodiments, the cannabinoid(s) component(s) of the modular system are associated (preferably by a health practitioner such as a natural-path or medical physician) with a treatment of the suspected or observed disease or disease symptom; without the enhancing or gain of function provided by the accessory module or the the combined system. In a non-limited example, the cannabinoid cannabidiol (CBD) is associated with treatment of inflammation; as such, the CBD containing system can be observed for treating inflammation, preferably for enhancing the treatment of inflammation in a subject (preferably a subject having an inflammation desirous of a treatment for inflammation, or recommended for treatment of inflammation by a health professional).

Certain embodiments of the present invention provide a cannabinoid composition including a cannabinoid and an accessory, wherein the composition has an enhanced water-solubility, binding affinity, targeting, biological activity, or any combination thereof; when compared to the cannabinoid apart from the accessory.

In preferred embodiments, the cannabinoid(s) refer to the compounds, and the structural and functional analogs thereof, in plants of the genus Cannabis, including marijuana and hemp; that interact with the mammalian endocannabinoid system in some manner including, but not limited to: binding, activating, inhibiting and/or modulating of factors including receptors, glycoproteins, enzymes, cellular proteins, membrane compounds, cellular compounds, intracellular compounds.

In certain embodiments, the cannabinoids includes: phytocannabinoids, endocannabinoids, analogs of phytocannabinoids, and analogs of endocannabinoids.

In certain embodiments, a composition or system of the present invention includes: an anti-microbial element, an anti-bacterial element, an anti-viral element, or a combination thereof. In certain preferred embodiments, the anti-microbial, anti-bacterial, anti-viral, or combination thereof comprises a peptide agent.

As used herein, the term cannabinoid refers to a group of chemical compounds, including certain fatty acid type compounds, that are structurally or functionally similar to the compounds of the endocannabinoid system. The term cannabinoid includes the phytocannabinoids which are observed in plants, the endocannabinoids that are observed in the endocannabinoid system of mammals (preferably humans, alternatively: livestock and companion animals).

Preferred cannabinoids herein include: phytocannabinoids (plant type), Cannabis (genus) derived phytocannabinoids, marijuana derived phytocannabinoids, hemp derived phytocannabinoids, synthetic phytocannabinoids, and synthetic phytocannabinoid analogs.

In preferred embodiments, the synthetic phytocannabinoids are chemical compounds that are observed in plants, but were chemically synthesized or biologically synthesized (e.g., a Cannabis type phytocannabinoid that is synthesized biologically in a heterologous system such as bacteria, yeasts or insect cells).

In certain preferred embodiments, a cannabinoid as used herein is an extract or isolate of a plant material (as opposed to synthetically manufactured by chemistry). (Isolates being cannabinoids purified or partially purified phytocannabinoids or synthetic analogs thereof.) Such may include: cannabinoid containing plant extracts, cannabinoid containing plant isolates (extracts that are enriched in the cannabinoids through purification or partial purification, enriching in the content of cannabinoids or by removal of non-cannabinoid components such as lipids, waxes, and carbohydrates).

Certain extracts and isolates of bioactive compounds from plants and methods therefor are given in Extraction, Isolation and Characterization of Bioactive Compounds from Plants' Extracts (Sasidharan et al., Afr J Tradit Complement Altern Med. (2011) 8(1):1-10), incorporated herein by reference in its entirety.

Certain embodiments provide preferred phytocannabinoids having a carboxylic functional group and includes one or more of: CBGA (Cannabigerolic acid), THCA (Δ9-tetrahydrocannabinolic acid), CBDA (Cannabidiolic acid), CBCA (Cannabichromenenic acid), CBGVA (Cannabigerovarinic acid), THCVA (Tetrahydrocanabivarinic acid), CBDVA (Cannabidivarinic acid), and CBCVA (Cannabichromevarinic acid).

The cannabinoids can be produced from plant biomass, an extract of a plant biomass, chemically synthesized cannabinoids preparations, biosynthesized cannabinoids, and other sources and preparations.

In certain embodiments, a composition, complex, system, or kit of the present invention includes a carboxyl group, such as the compound 11-nor-9-carboxy-Δ9-tetrahydrocannbinol, for example.

In certain embodiments, a stability enhancing additive is added to a composition, complex, system, or kit of the present invention to prevent oxidation including auto-oxidation (such as can occur with cannabidiol) and an exemplary such additive is citric acid. Other useful stabilizing factors include, but are not limited to: pH titration, light shielding, and temperature control at room temperature or maintenance at lower than room temperature.

Accessories

In certain embodiments, one or more accessory components is included in a composition, complex, system, or kit of the present invention; and examples of accessory components include, but are not limited to: a targeting moiety (passive, active, or both), a cyclodextrin, a poly-L-lysine, a hyaluronic acid, a hydrogel, a biomarker, a fluorescent label, a quantum dot, a flavor, or a combination of two or more of such components. In certain embodiments, the an accessory component is combined within a complex of the present invention by a covalent linkage, a non-covalent linkage, or both.

Peptides, polypeptides and proteins listed in DrugBank (version 5.1.2, released 2018 Dec. 20), incorporated herein by reference and useful for selecting water-soluble polypeptides and for selecting bioactive polypeptides. There are numerous bioactive peptide, polypeptides, and proteins that are commercially available, having characterized biological activities.

In preferred embodiments, a water-soluble accessory agent, such as a water-soluble polypeptide enhances the water-solubility of a cannabinoid component of a composition, complex, system, or kit of the present invention.

In certain embodiments, one or more accessory components is included in a composition, complex, system, or kit of the present invention and certain preferred embodiments, a component includes a terpene, a flavonoid, or both a terpene and a flavonoid. Exemplary terpenes include, but are not limited to: a beta-caryophyllene, an alpha-humulene, a caryophyllene oxide, a myrcene, an alpha-pinene, a terpinolene, a humulene epoxide, a beta-pinene, an e-beta-ocimene, and a linalool. Exemplary flavonoids include, but are not limited to: a vitexin, an apigenin, an orientin, a champhor, and a luteolin, a quercetin.

In certain embodiments; a composition, complex, system, or kit of the present invention includes, but is not limited to: a peptide, a receptor targeting peptide, an enzyme (an enzymatic polypeptide), an enzyme inhibitor (including polypeptide type enzymatic inhibitors), a biomarker targeting aptamer or polypeptide, or a combination of two or more thereof. In certain embodiments, such bioactive agent is a targeting moiety, an aptamer, a “RGD” element, a folic acid, a cell membrane penetrating polypeptide, a nuclear membrane penetrating polypeptide, a peptide nucleic acid (PNA, which, in certain embodiments include a sequence specificity for a selected DNA or RNA), or a combination of one or more thereof.

In certain embodiments; a composition, complex, system, or kit of the present invention includes, but is not limited to: a peptide nucleic acid sequence (PNA), a tag (e.g., a biomarker tag or purification tag), a cyclic polypeptide, a polypeptide having a disulfide bridge, a polypeptide including a branched chain amino acid, or any combination of two or more thereof.

In certain embodiments; a composition, complex, system, or kit of the present invention includes, but is not limited to: a release component, wherein the release component is separable for the cleavage or separation of components or modules of a system of the invention. Exemplary release components include, but are not limited to: a linkage that biodegrades in a cell, tissue, organ, or body. Certain examples of biodegradability are from changes in pH, ion concentration, temperature sensibility, and enzymatic attack.

In certain embodiments; a composition, complex, system, or kit of the present invention includes, but is not limited to: a porous delivery vehicle, which may include a mesoporous delivery vehicle.

Linkages

In certain preferred embodiments, the system modules are linked, preferably by an operable linkage. In certain preferred embodiments, the modules are operably linked and in more preferred embodiments, the operable linkage is reversible or can be cleaved or broken such that the modules can move completely independently. Certain embodied linkages, without being limiting, include: a bond, a covalent bond, a non-covalent bond, a hydrogen bond, an ionic bond, a binding interaction, a molecular interaction, an electrostatic attraction, and the like. In certain embodiments, the operable linkage is by containment in a confined space, preferably at or near the scale of the modular components of the system or on the nanoscale. In certain embodiments, a module of the system is a nanoscale container. Examples includes, but are not limited to, micro beads, nanoparticles, porous beads, polymers or silica particles having openings holes or binding elements such as aptamers, encapsulation, polymer encapsulation, lipid encapsulation, lipid membrane, or vesicle encapsulation), or combinations thereof, that contain other modules in proximity including by covalent or non-covalent bonding and by physical containment.

In certain embodiments, an operable linkage is a covalent linkage (having one or more covalent bonds holding components in association), non-covalent, or a combination of multiple types of bonds.

In certain embodiments, an operable linkage, or linker, includes one or more flexible units that confer a structural flexibility among linked modules, or rigidity units which provide stiffness to the assemble of components or a pair or a group of components, or both flexibility and rigidity linker components.

In certain embodiments are provided a method of extraction of cannabinoid from biomass such as with a carboxyl (COOH) reactive group that is water soluble, with a purification tag, or both. In certain embodiments, a carboxyl group is removed while adding to a carrier oil or other solubility factor.

Certain embodiments of the present invention provide systems, compounds, and methods for enhancing a property or activity of a cannabinoid.

Certain embodiments of the present invention; provide compounds, methods of use thereof and methods of manufacture; comprising: a small molecule chemical, preferably a phytocannabinoid, operably linked by an amide bond with a bioactive polymer. In certain preferred embodiments, the bioactive polymer enhances a property of a phytocannabinoid, provides a gain of function, or a combination thereof.

Certain preferred embodiments of the present invention provide compositions and methods of manufacture and use, comprising: polymer enhanced phytocannabinoids.

In certain embodiments, the instant compositions and methods relate to a compound, comprising: a phytocannabinoid linked by an amide bond to a polymer having a biological property, activity, or combination thereof.

In certain preferred embodiments, a phytocannabinoid, having a carboxyl functional group is operably linked with a polymer having an amide function group forming a polymer enhanced phytocannabinoid, a phytocannabinoid-polymer with an amide linkage. In certain preferred embodiments, the polymer enhanced phytocannabinoid has an increased water-solubility, an enhanced bioactivity, a gain of function, or a combination thereof.

In certain embodiments, a phytocannabinoid having a carboxyl functional group and a polymer (optionally a monomeric unit thereof) having an amide functional group are operably lined through chemical reaction forming a phytocannabinoid-polymer, the dash indicating that the phytocannabinoid and the polymer are linked by a covalent bond.

In certain optional embodiments, the phytocannabinoid is operably linked with an amino acid which is a monomeric unit of a polypeptide, which a type of polymer useful herein.

In certain embodiments herein, a phytocannabinoid-polymer (or, optionally, phytocannabinoid-amino acid) is referred to as a cannamide herein.

In certain embodiments herein, a cannamide may be described as a modular system, comprising a cannabinoid module (or component) and a polymer module (or component) combined (or operably linked) with a linker that is or includes an amide bond.

In certain embodiments, a cannamide is enhanced compared to a cannabinoid component without the benefit of the polymer.

Water-Soluble Cannamides (Enhanced Phytocannabinoids)

In certain embodiments, a cannamide has an enhanced solubility in a solvent. In certain preferred embodiments, the cannamide has an enhanced solubility in a water-based solvent (enhanced water-solubility property, compared to the cannabinoid in the absence of the linked polymer module). In certain embodiments, an enhanced water-solubility refers to a 15% or more enhancement, preferably a 20% or more enhancement, more preferably a 50% or more enhancement, and still more preferably, a 90% or more enhancement in water-solubility compared to the cannabinoid in absence of the linked polymer module.

In certain embodiments, the polymer module is selected for it's relative solubility in water, or aqueous-bases solutions. Water-soluble peptides (a biopolymer) and protein domains are useful for inclusion in a cannamide. Preferably, the water-solubility of the polymer module is 50% higher than the partner cannabinoid(s) and more preferably, two times or better yet three times more soluble.

In preferred embodiments, the term water-soluble refers to an amount or relative amount (e.g., on a percentage basis or as a ratio with the non-linked phytocannabinoid) of a compound that dissolves in water or a water-based solution. As is preferred herein, water solubility refers to the amount (or relative amount) dissolved as opposed to emulsions wherein a compound is in a carrier such as a micelle, liposome, or the like and does not truly dissolve, but is present in the solvent as an emulsion or a colloid. Thus, the emphasis herein on water-soluble cannamides is on dissolving and not emulsification. Emulsions are referred to herein as being water-compatible, but not being dissolved in the solute or solute solution. Issues observed with emulsions of cannabinoids include flocculation of the carrier lipids or lipid vesicles causing the emulsion particles to drop out of solution over time fouling the product.

Enhanced Bioactivity

In certain embodiments, a cannamide has an enhanced biological activity (bioactivity). In certain embodiments a cannamide is provided wherein the polymer module has an enhanced water-solubility (compared with the same type of phytocannabinoid without the operable linkage to the polymer in the same solvent and conditions).

Certain embodiments of the present invention make and provide water-soluble phytocannabinoids having a covalent linkage with a polymer, wherein the polymer includes a water-solubility enhancing moiety, a targeting moiety, a bioactive moiety, or one or more combination thereof.

Certain embodiments of the present invention make and provide a kit having two or more system components of the present invention and one or more reagents for combining thereof. In an exemplary embodiment, a kit is provided including: a phytocannabinoid, a water-soluble polypeptide, and a coupling reagent. Coupling reagents, including for amide bond formation are commercially available from suppliers. In certain embodiments, a kit of the present invention includes (in addition to the components above) a protecting group. Protecting groups are available from commercial suppliers and are useful, for example, to protect amino side-groups of amino acids in a polypeptide chain. In certain embodiments, a polypeptide is utilized or supplied with a kit wherein the polypeptide does not contain amino-type side-chain groups.

Certain embodiments of the present invention provide a therapeutic composition, comprising: a cannabinoid receptor agent and a cannabinoid enhancer. In certain embodiments, the composition further comprises a carrier suitable for delivery of the composition to a mammal, preferably a human in need of treatment with a cannabinoid for a disease or condition treatable with a cannabinoid.

The present invention provides conjugates of chemical compound having a carboxyl group operably linked with an amino acid or peptidic polymer via an amino bond.

Certain embodiments utilize a cannabinoid acid having a carboxyl group, examples of which include, but are not limited to: CBG-A, Cannabigerolic Acid, CBG, Cannabigerol, THC-A, Tetrahydrocannabinolic Acid, THC-C, Tetrahydrocannabinol-C4, THCV-A, Tetrahydrocannabivarinic Acid, A9THC, Delta-9-Tetrahydrocannabinol, A8THC, Delta-8-Tetrahydrocannabinol, THCV, Tetrahydrocannabivarin, CBN-A, Cannabinolic Acid, CBN, Cannabinol, CBD-A, Cannabidiolic Acid, CBDV-A, Cannabidivaric Acid, CBD, Cannabidiol, CBDV, Cannabidivarin, CBC-A, Cannabichromic Acid, CBC, Cannabichromene, CBC-A, Cannabicyclol Acid, or a combination of one or more thereof.

Amino acid, any of a group of organic molecules that consist of a basic amino group (—NH2), an acidic carboxyl group (—COOH), and an organic R group (or side chain) that is unique to each amino acid. The term amino acid is short for α-amino [alpha-amino] carboxylic acid. Each molecule contains a central carbon (C) atom, called the α-carbon, to which both an amino and a carboxyl group are attached. The remaining two bonds of the α-carbon atom are generally satisfied by a hydrogen (H) atom and the R group.

The term bioCBX herein refers to a cannabinoid, preferably a phytocannabinoid, having an enhanced bioactivity, a gain of bioactive function, or both.

Methods

Extractions

Certain embodiments provide a method of extracting cannabinoids from cannabinoid producing plants, comprising: reacting a water-soluble peptide with a plant biomass under conditions for amide bond formation between complementary amino and carboxylic functional groups on the cannabinoids and the polypeptides and performing aqueous and organic extraction steps to collect cannabinoids separate from the matrix.

Certain embodiments providing a targeting moiety operably linked with a cannabinoid and certain useful targeting agents include, but are not limited to: a cell type recognition agent, a diagnostic marker, a disease treating agent (e.g., for treating dementia or for treating a cancer such as by killing cancer cells or killing dividing cells or stopping cell division). In certain embodiments, a targeting moiety includes a membrane penetrating component, a nucleus penetrating component, an endoplasmic reticulum penetrating component, a lysosome penetrating component, a Golgi penetrating component, or a combination of more than one of such components. In certain embodiments, the instant components are described as cell or organelle concentrating components (in alternative to penetrating components).

In certain embodiments, a targeting component concentrated or targets a composition, complex, or system of the present invention to an organ, including, but not limited to the brain, liver, or nerve cell (peripheral, central, or both); or a combination thereof.

Cleavage of an Amino Moiety

Certain embodiments provide a method of forming a cannabinoid-amino acid compound by reacting together a phytocannabinoid having a carboxylic acid group and an amino acid having an alpha-amino group via a condensation reaction.

Certain embodiments provide a method of forming a cannabinoid-polymer compound by reacting together a phytocannabinoid having a carboxylic acid group and a polymer having an alpha-amino group, preferably forming a cannabinoid-polymer hybrid compound operably linked by an amino bond, most preferably an alpha-amino bond. In preferred embodiments, moieties (the components of the compound) are combined via a condensation reaction.

Certain preferred uses of a compound of the present invention include enhancing the water-solubility of a phytocannabinoid by covalently linking the phytocannabinoid with a water-soluble module comprising a water-soluble amino acid or alpha-amino polymer.

In organic chemistry, functional groups are specific substituents or moieties within molecules that are responsible for the characteristic chemical reactions of those molecules. The same functional group will undergo the same or similar chemical reaction(s) regardless of the size of the molecule it is a part of.[1][2] This allows for systematic prediction of chemical reactions and behavior of chemical compounds and design of chemical syntheses. Furthermore, the reactivity of a functional group can be modified by other functional groups nearby. In organic synthesis, functional group interconversion is one of the basic types of transformations.

Functional groups are groups of one or more atoms of distinctive chemical properties no matter what they are attached to. The atoms of functional groups are linked to each other and to the rest of the molecule by covalent bonds. For repeating units of polymers, functional groups attach to their nonpolar core of carbon atoms and thus add chemical character to carbon chains. Functional groups can also be charged, e.g. in carboxylate salts (—COO—), which turns the molecule into a polyatomic ion or a complex ion. Functional groups binding to a central atom in a coordination complex are called ligands. Complexation and solvation are also caused by specific interactions of functional groups. In the common rule of thumb “like dissolves like”, it is the shared or mutually well-interacting functional groups which give rise to solubility. For example, sugar dissolves in water because both share the hydroxyl functional group (—OH) and hydroxyls interact strongly with each other. Plus, when functional groups are more electronegative than atoms they attach to, the functional groups will become polar, and the otherwise nonpolar molecules containing these functional groups become polar and so become soluble in some aqueous environment.

In certain embodiments; a composition, complex, system, or kit of the present invention includes, but is not limited to: a single cannamide (without a second type of cannamide present).

In certain embodiments; a composition, complex, system, or kit of the present invention includes, but is not limited to: a mixture of two or more types of cannamides.

Three-dimensional, donut-shaped cyclodextrins have a hydrophobic cavity on the inside and a hydrophilic cover on the outside and are useful in certain embodiments, especially for confinement of a composition, complex, system, or kit of the present invention.

In certain embodiments, a containment substance is used to effect a delayed or time sensitive release of a cannamide or component thereof, of the present invention, wherein containment substance is a partial barrier or includes an operable gate for containing the composition and dispensing a composition, complex, or system described herein. In certain embodiments, the containment substance, comprises: a hydrogel, a glucomannan, a polysaccharide, glycoside, cyclodextrin, a microbead, a crosslinked polymer network, or any combination thereof.

Certain embodiments provide a composition, comprising: a water-soluble particle (e.g., a microparticle, a microbead, or a networked linking agent) containing a cannabinoid and a targeting polypeptide, a bioactive polypeptide or both (preferably, when a targeting peptide is included, it is exposed at the surface of the particle or has access or exchange with the surface thereof).

Certain embodiments provide an aqueous preparation of a Cannabis biomass, comprising: a solvated complex including a phytocannabinoid, or an acid thereof, and a water-soluble polypeptide, preferably an isolate of a Spirulina, and more preferably an SP6 peptide isolate of the Spirulina (preferably, Spirulina platensis).

Certain embodiments provide a composition, comprising: an aqueous preparation of marijuana or hemp biomass including a solvated complex, the complex including a phytocannabinoid and a water-solubility domain, preferably a water-soluble polypeptide which enhances the water-solubility of the complex.

### EXAMPLES

Certain embodiments of the present invention are provided in non-limiting examples herein.

### Example 1

This example includes a table of non-limiting exemplary embodiments of combinations of cannabinoids/phytocannabinoids linked with solubility polypeptides and is provided for reference. Certain preferred embodiments include solubility polypeptide modules having hydrophilic enhancing features.

Table for Example 1

| Exemplary | Cannabinoid | Bioactive | Linkage | Preferred |
|---|---|---|---|---|
| Compositions | Module | Module | (includes) | Use(s) |
| Composition A | One or more | One hydrophilic | Covalent amide | Enhanced |
| | cannabinoids | amino acid | bond | water-solubility |
| | | | | Research tool |
| Composition A2 | One cannabinoid | One hydrophilic | Covalent amide | Enhanced |
| | | polypeptide | bond | water-solubility |
| | | | | Research tool |
| Composition B | One or more | One or more | Covalent amide | Enhanced |
| | phytocannabinoids | hydrophilic amino | bond | water-solubility |
| | | acids | | Research tool |
| Composition B1 | A phytocannabinoid | A hydrophilic | Covalent amide | Enhanced |
| | | polypeptide | bond | water-solubility |
| | | | | A water-based |
| | | | | CBX remedy/beverage |
| | | | | Research tool |
| Composition C | CBD(A) | A hydrophilic | Covalent amide | Enhanced |
| | | amino acid or | bond | water-solubility |
| | | polypeptide | | An aqueous CBD |
| | | | | remedy/beverage |
| Composition C1 | THC(A) | A hydrophilic | Covalent amide | Enhanced |
| | | amino acid or | bond | water-solubility |
| | | polypeptide | | An aqueous THC |
| | | | | remedy/beverage |
| Composition C2 | CBG(A) | A hydrophilic | Covalent amide | Enhanced |
| | | amino acid or | bond | water-solubility |
| | | polypeptide | | An aqueous CBG |
| | | | | remedy/beverage |
| Composition C3 | A phytocannabinoid | A hydrophilic | Covalent amide | Enhanced |
| | extract or isolate of | amino acid or | bond | water-solubility |
| | marijuana | polypeptide | | Water-based marijuana |
| | | | | remedy/beverage |
| Composition C4 | A phytocannabinoid | A hydrophilic | Covalent amide | Enhanced |
| | extract or isolate of | amino acid or | bond | water-solubility |
| | hemp | polypeptide | | Water-based hemp |
| | | | | remedy/beverage |
| Composition D | One or more | One or more | Covalent amide | Enhanced |
| | cannabinoids | amphiphilic | bond | water-solubility |
| | | polypeptides | | of the composition |
| | | | | Enhanced emulsions |
| Composition E | A phytocannabinoid | One or more | Covalent amide | Enhanced |
| | | hydrophilic | bond | water-solubility |
| | | polypeptides | | A water-based CBX |
| | | having determined | | remedy/beverage |
| | | sequence(s) | | |
| Composition F | A phytocannabinoid | A hydrophilic | Covalent amide | Enhanced |
| | | domain having an | bond | water-solubility |
| | | amine group (for i | | A water-based CBX |
| | | linkage) | | remedy/beverage |
| Composition F | A phytocannabinoid | A hydrophilic | Covalent amide | Enhanced |
| | | domain having a | bond | water-solubility |
| | | carboxyl group (for | | A water-based CBX |
| | | linkage) | | remedy/beverage |
| Composition G | A phytocannabinoid | A bioactive | Covalent amide | Enhanced |
| | | polypeptide having | bond | water-solubility |
| | | an amine group | | A water-based CBX |
| | | (for linkage) | | remedy/beverage |
| Composition G1 | A phytocannabinoid | A hydrophilic | Covalent amide | Enhanced |
| | | domain having a | bond | water-solubility |
| | | carboxyl group (for | | A water-based CBX |
| | | linkage) | | remedy/beverage |
| Composition H | A phytocannabinoid | A bioactive | Covalent amide | A research tool |
| | | polypeptide | bond | A bioCBX remedy |
| Composition H1 | A phytocannabinoid | A bioactive | Covalent amide | A research tool |
| | | polypeptide | bond | A bioCBX remedy |
| | | (nerve cell binding) | | |
| Composition H2 | A phytocannabinoid | A bioactive | Covalent amide | A research tool |
| | (first | polypeptide | bond | A bioCBX remedy |
| | endocannabinoid | (second | | |
| | receptor binding | endocannabinoid | | |
| | feature) | receptor binding | | |
| | | feature) | | |
| Composition H3 | A phytocannabinoid | A bioactive | Covalent amide | A research tool |
| | | polypeptide | bond | A bioCBX remedy |
| | | (cell penetrating | | |
| | | feature) | | |
| Composition H4 | A phytocannabinoid | A bioactive | Covalent amide | A research tool |
| | | polypeptide | bond | A bioCBX remedy |
| | | (nuclear location | | |
| | | feature) | | |
| Composition H5 | A phytocannabinoid | A bioactive | Covalent amide | A research tool |
| | | polypeptide | bond | A bioCBX remedy |
| | | (membrane | | |
| | | transport module | | |
| | | and a nuclear | | |
| | | location module) | | |
| Composition H6 | A phytocannabinoid | A bioactive | Covalent amide | A research tool |
| | | polypeptide | bond | A bioCBX remedy |
| | | (enzyme inhibitor | | |
| | | feature) | | |
| Composition H7 | A phytocannabinoid | A bioactive | Covalent amide | A research tool |
| | | polypeptide | bond | A bioCBX remedy |
| | | (an FAAH enzyme | | |
| | | inhibitor feature) | | |
| Composition H7 | A phytocannabinoid | A bioactive | Covalent amide | A research tool |
| | | polypeptide | bond | A bioCBX remedy |
| | | (an FAAH enzyme | | |
| | | inhibitor feature) | | |
| Composition I | A phytocannabinoid | A hydrophilic | Covalent amide | A research tool |
| | | domain module | bond | A bioCBX remedy |
| | | AND a bioactive | | |
| | | polypeptidemodule | | |

Table notes:
As set forth herein, the terms “a” and “an” in describing elements of the present invention and claim elements mean “one or more.”
A phytocannabinoid modules (one or more), optionally includes synthetic analogs of phytocannabinoids.
Enhanced water-solubility refers to the composition compared to the cannabinoid(s) thereof, but in the absence of the non-cannabinoid modules (the cannabinoid(s) apart from the composition), with a preferred enhancement of 15% or more.
Preferred cannabinoids in the example of the table are phytocannabinoids, optionally analog(s) of a phytocannabinoid or synthetic phytocannabinoid(s). Certain preferred cannabinoids exclude endocannabinoids (alternatively, have an insignificant amount of endocannabinoid(s) present).
A bioCBX refers to a cannabinoid, preferably a phytocannabinoid, having an enhanced bioactivity, a gain of bioactive function, or both.
A preferred amide bond is an alpha amide bond type with the bond to an alpha amino acid (solo or a terminal alpha amino acid of a polypeptide).
The term CBX refers to the cannabinoids, in general. That is one or more types of cannabinoids wherein the “X” in CBX refers to a wildcard. In the example shown in the table, CBX preferably refers to the phytocannabinoids and analogs thereof (alternatively not including endocannabinoids).
A hydrophilic polypeptide includes charged and polar amino acids rendering the polypeptide water-soluble (e.g., 5 mg/ml or more dissolved in water or an aqueous solution, additional amounts disclosed herein).
Composition H: certain embodiments include one or more bioactive polypeptides preferably having a feature/property of: targeting, binding, activating, inhibiting, penetrating, concentrating, transporting, or any combination thereof.

### Example 2

This example includes a table of non-limiting exemplary embodiments of combinations of phytocannabinoids linked with bioactive polypeptides. The composition I includes one or more phytocannabinoid modules, a hydrophilic module (for water-solubility enhancement) and a bioactive polypeptide module featuring enhanced bioactivity or a gain-of-function, being a function not observed or characterized for the phytocannabinoid module(s).

Table for Example 2

| Exemplary | Cannabinoid | Bioactive | Linkage | Preferred |
|---|---|---|---|---|
| Compositions | Module | Module | (includes) | Use(s) |
| Composition H | A phytocannabinoid | A bioactive | Covalent amide | A research tool |
| | | polypeptide | bond | A bioCBX remedy |
| Composition H1 | A phytocannabinoid | A bioactive | Covalent amide | A research tool |
| | | polypeptide | bond | A bioCBX remedy |
| | | (nerve cell binding) | | |
| Composition H2 | A phytocannabinoid | A bioactive | Covalent amide | A research tool |
| | (first | polypeptide | bond | A bioCBX remedy |
| | endocannabinoid | (second | | |
| | receptor binding | endocannabinoid | | |
| | feature) | receptor binding | | |
| | | feature) | | |
| Composition H3 | A phytocannabinoid | A bioactive | Covalent amide | A research tool |
| | | polypeptide | bond | A bioCBX remedy |
| | | (cell penetrating | | |
| | | feature) | | |
| Composition H4 | A phytocannabinoid | A bioactive | Covalent amide | A research tool |
| | | polypeptide | bond | A bioCBX remedy |
| | | (nuclear location | | |
| | | feature) | | |
| Composition H5 | A phytocannabinoid | A bioactive | Covalent amide | A research tool |
| | | polypeptide | bond | A bioCBX remedy |
| | | (membrane | | |
| | | transport module | | |
| | | and a nuclear | | |
| | | location module) | | |
| Composition H6 | A phytocannabinoid | A bioactive | Covalent amide | A research tool |
| | | polypeptide | bond | A bioCBX remedy |
| | | (enzyme inhibitor | | |
| | | feature) | | |
| Composition H7 | A phytocannabinoid | A bioactive | Covalent amide | A research tool |
| | | polypeptide | bond | A bioCBX remedy |
| | | (an FAAH enzyme | | |
| | | inhibitor feature) | | |
| Composition H7 | A phytocannabinoid | A bioactive | Covalent amide | A research tool |
| | | polypeptide | bond | A bioCBX remedy |
| | | (an FAAH enzyme | | |
| | | inhibitor feature) | | |
| Composition I | A phytocannabinoid | A hydrophilic | At least one | A research tool |
| | | domain module | covalent amide | A bioCBX remedy |
| | | AND | bond between a | |
| | | a bioactive | phytocannabinoid | |
| | | polypeptide | module and the | |
| | | module | hydrophilic | |
| | | | module, bioactive | |
| | | | module, or both | |

### Example 2

In certain embodiments, a bioactive module includes a feature or property that enhances a similar activity of the CBX partner in the composition (example, binding to a CB1 or CB2 receptor). For example, a THCA module includes a CB1 binding feature and a synaptic targeting polypeptide module includes a feature of concentrating at nerve synapses. Certain embodiments of the present invention provide a composition comprising a THCA module linked with a synaptic targeting polypeptide module by an amide bond. In certain preferred embodiments, the combination has an enhanced binding to CB1 receptors present in nerve synapses.

In certain embodiments, a bioactive module includes a feature or property that enhances a similar activity of the CBX partner in the composition.

### Example 3

The modules of the present invention including those in the examples can be “mixed and matched.” Each combination of modules provides a research tool or reagent for measuring properties of the module composition. In certain embodiments, the measured properties are compared to expected module features, such as the named, assigned or predicted module function. Such studies are useful, for example, in identifying structural and/or functional domains of the cannabinoids critical to their biological activity or activities. In certain embodiments, a cannabinoid is combined with a biologically active module, a water solubility module, or both are referred to as: bioCBX, aquaCBX and/or bioaqua-CBX compositions, respectively.

Isotonic Preparations

In certain embodiments, composition, complex, system, or kit of the present invention is provided in an isotonic oral delivery formulation.

In certain embodiments, composition, complex, system, or kit of the present invention is provided in a carbonated drink or beverage.

Twenty naturally occurring amino acids in humans are giving herein, grouped by certain properties. The three letter and one letter designations for each amino acids are included.

| Side Chain Characteristic | Amino Acids |
|---|---|
| Charged (side chains often form salt bridges) | Arginine - Arg - R |
| (Hydrophilic side chains) | Lysine - Lys - K |
| | Aspartic acid - Asp - D |
| | Glutamic acid - Glu - E |
| Polar | Glutamine - Gln - Q |
| (Hydrophilic side chains) | Asparagine - Asn - N |
| | Histidine - His - H |
| | Serine - Ser - S |
| | Threonine - Thr - T |
| | Tyrosine - Tyr - Y |
| | Cysteine - Cys - C |
| Amphipathic (often observed at the surfaces of | Tryptophan - Trp - W |
| proteins or lipid membranes, sometimes also | Tyrosine - Tyr - Y |
| classified as polar(e.g., Met)) | Methionine - Met - M |
| Hydrophobic | Alanine - Ala - A |
| | Isoleucine - Ile - I |
| | Leucine - Leu - L |
| | Methionine - Met - M |
| | Phenylalanine - Phe - F |
| | Valine - Val - V |
| | Proline - Pro - P |
| | Glycine - Gly - G |

Methionine is alternatively considered to be amphipathic and hydrophobic.

What is claimed is:

1. A composition, comprising: a cannamide solvated in an aqueous solution.

2. The composition of claim 1, wherein the cannamide includes a cannabinoid, a water-soluble polypeptide and a linker having a covalent amide bond operably linking the cannabinoid and the polypeptide.

3. The composition of claim 1, wherein the cannamide comprises an operable linkage of a cannabinoid and a water-soluble polypeptide, wherein the operable linkage includes a non-covalent binding interaction.

4. The composition of claim 1, further comprising a containment substance including a cannamide having a cannabinoid and a water-soluble polypeptide operably linked by an inclusion in the containment substance.

5. The composition of claim 4, wherein the containment substance comprises a semipermeable barrier for containing the composition or component thereof.

6. The composition of claim 1, further comprising a containment substance including a cannamide having a cannabinoid and a water-soluble polypeptide operably linked a containment in the containment substrate.

7. The composition of claim 1, wherein the cannamide comprises one or more cannabinoids operably linked with a water-soluble polypeptide and a bioactive polypeptide.

8. The composition of claim 1, wherein the cannamide comprises one or more cannabinoids operably linked with a water-soluble polypeptide and the composition includes one or more bioactive agents selected from the group consisting of: a polypeptide, a phytocannabinoid, a non-cannabinoid chemical agent, a peptide nucleic acid (PNA), a deoxyribonucleic acid (DNA), a ribonucleic acid (RNA), an aptamer, an antibody, a membrane transport sequence, a nuclear localization sequence, a lipid, a phospholipid, an anandamide, an angiogenesis inhibitor, an RGD containing polypeptide, a fluorescent molecule, a fluorescent nanoparticle, a quantum dot, a toxin, a chemotherapeutic, or any combination thereof.

9. The composition of claim 1, wherein the cannamide includes the one or more bioactive agents.

10. The composition of claim 1, further comprising a terpene, a flavonoid, a bioactive polypeptide, a bioactive lipid, a bioactive non-cannabinoid compound, or any combination thereof.

11. The composition of claim 1, wherein the cannabinoid is a cannabigerol (CBG), a cannabigerolic acid (CBGA), a tetrahydrocannabinol (THC) a tetrahydrocannabiolic acid (THCA), a cannabidiol (CBD), a cannabidiolic acid (CBDA), a cannabinol (CBN), a cannabinolic acid (CBNA) or any combination thereof.

12. The composition of claim 1, wherein the cannamide includes a bioactive agent isolated from a biomass of Spirulina.

13. The composition of claim 1, wherein the composition does not include a significant amount of a surfactant.

14. The composition of claim 1, further comprising a bioactive polypeptide having a feature or a property of modulating: a binding activity, a receptor activity, an enzymatic activity, a concentrating activity, a localization activity, a penetrating activity, a transporting activity, a marking activity, a toxic activity, an activity, a reduction activity, an oxidation activity, an inhibition of oxidation activity, an absorption activity, or any combination thereof.

15. The composition of claim 14, comprising two or more bioactive polypeptides.

16. A composition, comprising: a water-soluble particle containing a cannabinoid and a targeting polypeptide, a bioactive polypeptide or both.

17. An aqueous preparation of a Cannabis biomass, comprising: a solvated complex including a phytocannabinoid, or an acid thereof, and a water-soluble polypeptide.

18. The preparation of claim 17, where complex includes one or more of a cannabigerol (CBG), a cannabigerolic acid (CBGA), a tetrahydrocannabinol (THC) a tetrahydrocannabiolic acid (THCA), a cannabidiol (CBD), a cannabidiolic acid (CBDA), a cannabinol (CBN), a cannabinolic acid (CBNA) or any combination thereof.

19. The preparation of claim 17, wherein the water-soluble polypeptide is a protein hydrolysate.

20. The preparation of claim 17, wherein the water-soluble polypeptide is a hydrolysate of a silk protein, a casein protein, a branched chain amino acid preparation, a whey protein, a wheat protein, a soy protein, a collagen protein, a fish protein, blue-green algae protein, a Spirulina, or any combination thereof.
